(19) United States
(12) Patent Application Publication
(10) Pub. No.: US 2008/0199430 A1
(43) Pub. Date: Aug. 21, 2008

(54) Methods and kits for qualifying sperm cells

(75) Inventor: Shafrira Shai, Kiryat-Haim (IL)

Correspondence Address: Martin D. Moynihan;PRTSI, Inc., P.O. Box 16446, Arlington, VA, 22215, US

(73) Assignee: Dyn-BioShaf (2006) Ltd., Caesarea (IL)

(21) Appl. No.: 11/707,126

(22) Filed: Feb. 16, 2007

(51) Int. Cl. C12N 5/06; C12Q 1/00

(52) U.S. Cl. 424/93.1; 435/366; 435/4

(57) ABSTRACT

A method of qualifying sperm cells is provided. The method comprising: (a) staining the sperm cells with acridine orange so as to obtain stained sperm cells; and (b) using a fluorescence activated cell sorter (FACS), gating events simultaneously stained with a green fluorescence intensity higher than 100 nm and red fluorescence intensity in a range selected from 20-1010 nm, the events representing a cell population of the stained sperm cells, wherein a percentage of the cell population is indicative of the quality of the sperm cells.

### FIELD AND BACKGROUND OF THE INVENTION

The present invention relates to methods and kits for qualifying sperm cells.

Infertility affects 10-18% of couples in industrially developed countries and it is likely that the global deterioration of semen parameters (most pronounced in industrialized societies), will increase these percentages.

Semen analysis is a crucial, primary test for both identifying male infertility and determining its causes. Assessment of sperm quality is also an important procedure for evaluating effects of drugs on testis function in laboratory animals. Conventional semen analysis uses quantitative as well as qualitative parameters. Traditionally, laboratory experts perform semen analysis using a light microscope, with either a fresh or stained sample, in accordance to the manual of the W.H.O [World Health Organization. Laboratory manual for the examination of human semen and sperm-cervical mucus interaction. 4^{th} ed. New York: Cambridge University Press, 1999]. This analysis includes a manual assessment of physical and morphological characteristics for semen and sperm, sperm concentration, sperm vitality, sperm auto-antibodies and presence and number of White Blood Cells and Round cells.

However, a significantly different fertilization rate has been reported for patients with similar semen parameters, suggesting that another diagnostic marker is needed to identify the inherited defects which render certain spermatozoa unable to fertilize or result in good pregnancy outcome [Wolf D P, J Assist Reprod Genet 10: 246-50 (1993)]. In addition, specific medical procedures require different sperm characteristics [e.g., in vitro fertilization (IVF) requires integrity of DNA but not necessarily sperm motility]. Thus, there is a need for further methods for assessing sperm samples.

The integrity of sperm nuclei and DNA is a crucial factor in the success of fertilization and pregnancy. Great deal of evidence suggests that complete chromatin condensation and packaging is essential for normal sperm function, and is an indicator of DNA integrity [Kosower et al., J Androl 13:342-8 (1992); Manicardi G C et al., Biol Reprod 52:864-7(1995)]. The association between chromatin condensation of sperm cells and in vitro fertilization rate is well known [Hammadeh M E et al., Andrologia 30(1):29-35 (1998); Witt et al., Human Reprod 18:O-022 (2003)].

Sperm morphology has been conventionally used as an indicator for sperm with abnormal DNA [(Fawcett D W et al., Dev Biol 26: 220-515 (1971)]. However, it is difficult, if not practically impossible, to distinguish variations of chromatin condensation by light microscopy usually employed in screening laboratories. Studies have demonstrated that male factor infertility patients possess hidden anomalies in the composition of their sperm nuclei, displaying a higher level of loosely packaged chromatin and damaged DNA [Evenson D P et al., Cytometry 7:45-53 (1986)]. In another example, a comparison between microscopic morphological analysis and a computerized karyometric image analysis (CKIA) of sperm samples taken for ICSI, it was found that DNA analysis is better predictive of sperm cell quality than standard morphology [RAMOS et al., J. androl 2004, vol. 25, no 3, pp. 406-411]

Thus, some spermal DNA integrity and spermal DNA condensation defects, which are crucial for determining successful fertilization, may not be detected through conventional microscopic analysis.

In addition, due to the nature of the conventional assay, there is a high percentage of inter- and intra-laboratory variability for both the evaluation of morphology and in the assessment of quantitative parameters such as sperm concentration [Matson, P. L., Hum Reprod 1995; 10:620-625]. Besides the subjective nature of human evaluation, which depends on the experience and competency of the observer, human observers typically assess a few hundred cells in a population of millions, which may lead to an incorrect reflection of the whole sample. Application of a rapid, automated and precise method for the evaluation of semen quality, which is specifically associated with in vitro fertilization and pregnancy success, is therefore advantageous in many aspects.

Recently, flow cytometry technology has been used to study diagnostic parameters such as reproductive capacity. The Flow Cytometer (FC) has been developed such that it is cost-effective for diagnostic functions, with an output far exceeding that obtained using a microscope. Flow cytometry offers speed, accuracy, and precision that may be overlooked using other techniques. Recently, flow cytometry has been applied to evaluate sperm cell membrane integrity, mitochondrial function, acrosomal status, chromatin structure, cation concentrations, sperm-bound antibodies and chromosomal analysis.

At present, flow cytometry methods are used for assessing sperm cell properties such as concentration, morphology and viability. Among other uses, flow cytometry is used with fluorescent DNA interacting reagents for sperm cell analysis:

Christensen et al. [J. Andrology, 2004 25(2):255-64] teach a method of gate analysis of sperm cell concentration and viability using fluorescent beads. The method is mainly used for analyzing high sperm concentration (in boar and bull).

U.S. Patent Application 20060257909 teaches the use of a vital DNA stain to distinguish between sperm containing X and Y chromosomes.

Acridine orange is an intercalating agent used for staining DNA. Different DNA properties cause the fluorescent emission by this dye to vary between red and green. For example, single stranded and double stranded DNA emit red and green fluorescence, respectively when dyed with acridine orange. Use of the acridine orange fluorescence range for analyzing chromatin condensation was taught by U.S. Pat. No. 4,559,309. However, as stated in the patent, the relationship between chromatin condensation and fertility is unclear.

U.S. Patent Application 20050009115 teaches analysis of sperm morphology using an acridine orange staining pattern, which defines normal DNA morphology.

None of the methods described hereinabove relate to the use of an acridine orange staining pattern as an indicator of normal DNA condensation, characteristic of fertile sperm.

There is thus a widely recognized need for, and it would be highly advantageous to have highly accurate flow cytometry based methods, to evaluate sperm quality in biological samples.

### SUMMARY OF THE INVENTION

According to one aspect of the present invention there is provided a method of qualifying sperm cells, the method comprising staining the sperm cells with acridine orange so as to obtain stained sperm cells; and using a fluorescence activated cell sorter (FACS); gating events simultaneously stained with a green fluorescence intensity higher than 100 nm and red fluorescence intensity in a range selected from 20-1010 nm, the events representing a cell population of the stained sperm cells, wherein a percentage of the cell population is indicative of the quality of the sperm cells.

According to another aspect of the present invention there is provided a method of collecting a sperm cell population, the method comprising staining sperm cells with acridine orange so as to obtain stained sperm cells; using a fluorescence activated cell sorter (FACS), gating events simultaneously stained with a green fluorescence intensity higher than 100 nm and red fluorescence intensity in a range selected from 20-1010 nm, the events representing a cell population of the stained sperm cells; and collecting the cell population of the stained sperm cells.

According to yet another aspect of the present invention there is provided a method of improving pregnancy outcome, the method comprising staining sperm cells with acridine orange so as to obtain stained sperm cells; using a fluorescence activated cell sorter (FACS) gating events simultaneously stained with a green fluorescence intensity higher than 100 nm and red fluorescence intensity in a range selected from 20-1010 nm, the events representing a cell population of the stained sperm cells; collecting the cell population of the stained sperm cells; and using the cell population for fertilization, thereby improving pregnancy outcome.

According to further features in preferred embodiments of the invention described below, when the cell population is above 65% of the stained sperm cells the quality of the sperm cells is good.

According to still further features in the described preferred embodiments the fertilization is effected by a method selected from the group consisting of artificial insemination and in vitro fertilization (IVF).

According to still another aspect of the present invention there is provided an isolated sperm cell population generated according to the method.

According to an additional aspect of the present invention there is provided a kit for qualifying sperm cells, the kit comprising acridine orange; and instructions for analyzing acridine orange stained sperm cells simultaneously stained with a green fluorescence intensity higher than 100 nm and red fluorescence intensity in a range selected from 20-1010 nm; gated by FACS.

According to still further features in the described preferred embodiments the cell population is characterized by a normal DNA condensation pattern.

According to still further features in the described preferred embodiments the sperm cells are of a mammalian semen sample.

According to still further features in the described preferred embodiments the sperm cells are of a human semen sample.

The present invention successfully addresses the shortcomings of the presently known configurations by providing flow cytometry based methods for assessing the quality of a sperm sample as well as for the collection of good quality sperm from an individual sample for the purpose of fertilization, based on the DNA condensation of the sperm.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the Drawings:

FIGS. 1a-d show acridine orange-stained sperm samples exhibiting simultaneous staining intensity as follows: green fluorescence higher than 100 nm and a red fluorescence between 20 nm-1010 nm. Cells falling within the gate are designated normal. A sperm sample is considered normal when above 65% of the cells fall within the aforementioned gate. FIG. 1a depicts staining pattern of a normal sperm sample (86.38% of events occurring within the gate). FIG. 1b depicts an abnormal sperm sample (54.39% of events occurring within the gate). FIG. 1c depicts an untreated normal sperm sample (73.46% of events occurring within the gate). FIG. 1d is a heat treated (70° C., for 2 hours), sperm sample (46.6% of events occurring within the gate).

### DESCRIPTION OF THE PREFERRED EMBODIMENTS

The present invention is of methods and kits for qualifying sperm cells and use thereof in sperm cell collection and improving pregnancy outcome.

The principles and operation of the qualification method according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Semen analysis is usually performed with the aid of manual techniques and microscopic evaluation by laboratory operators. The test is tedious, expensive, time consuming and not standardized, influencing its clinical validity.

The use of flow cytometry methodology for sperm cell qualification is meant to address these inaccuracies and low output permitting the analysis of many millions of cells in few seconds.

Acridine orange is an intercalating agent used for staining DNA. Different DNA properties cause the fluorescent emission by this dye to vary between red and green. For example, single stranded and double stranded DNA emit red and green fluorescence, respectively when dyed with acridine orange.

Use of the acridine orange fluorescence range for analyzing chromatin condensation in sperm cells was taught by U.S. Pat. No. 4,559,309. However, this patent states the relationship between chromatin condensation and fertility is unclear.

U.S. Pat. Application No. 20050009115 teaches analysis of sperm morphology using an acridine orange staining pattern. However, a specific gate which qualifies sperm on the basis of DNA condensation, characteristic of normal sperm is not described.

While reducing the present invention to practice the present inventor has designed a novel gate for characterizing sperm cells with a normal DNA condensation pattern. This gate can be used for the accurate, single step characterization of normal sperm cells which, in turn, can be collected and used in fertilization to improve pregnancy outcome.

As is illustrated in the examples section which follows, the present inventor has preformed a laborious clinical study on three hundred semen samples obtained from fertile males categorized as having normal sperm morphology, and one hundred semen samples analyzed as having an abnormal sperm's head morphology, according to the latest manual of the W.H.O [World Health Organization. Laboratory manual for the examination of human semen and sperm-cervical mucus interaction. 4^{th} ed. New York: Cambridge University Press, 1999]. On the basis of the Acridin Orange staining pattern of DNA from normal sperm, a novel gate for normal sperm head morphology, which is indicative of DNA condensation, was elucidated. A good quality sample was hence characterized as having at least 65% sperm cells within the newly uncovered gate.

Thus, according to one aspect of the present invention, there is provided a method of qualifying sperm cells. The method comprising staining the sperm cells with acridine orange so as to obtain stained sperm cells; and using a fluorescence activated cell sorter (FACS), gating events simultaneously stained with a green fluorescence intensity higher than 100 nm and red fluorescence intensity in a range selected from 20-1010 nm, said events representing a cell population of said stained sperm cells, wherein said number of events is indicative of the quality of the sperm cells.

As used herein the phrase “sperm cells” refers to sperm cells of any mammalian subject (e.g., bull) preferably a human subject. The sperm cells may be part of a crude semen sample or somewhat diluted as will be further described hereinbelow.

As used herein the phrase “qualifying sperm cells” refers to assessing the ability of the sperm cells to fertilize and/or improve pregnancy outcome.

As mentioned hereinabove, a semen sample may be from the ejaculate of any male mammal. As semen samples are relatively viscous, it is normally preferred that the semen sample is diluted. The dilution of the sample is preferably performed using a diluent which sustains viability and motility of the sperm cells during the determination, such as phosphate-buffered saline with nutrient mixture (e.g., Ham's F-10 Buffer, see Examples section)

In order to obtain an even higher degree of accuracy, automated dilution of the semen may be applied. Thereby, the operator-dependant part of the process is eliminated which makes the process highly reproducible.

In order not to compromise sperm vitality as a result of dilution or freezing/thawing procedures, semen samples may be collected and preserved in Media containing TEST-yolk buffer (TYB), which has been shown to be effective in preserving sperm when refrigerated for short intervals of time, and for long term frozen storage with the addition of glycerol. For IUI (Intra Uterine Insemination), IVF (In Vitro Fertilization) and other assisted reproductive procedures, medium with glycerol, but without TEST-yolk buffer, may be used for cryopreservation and storage. Examples of sperm preservation mediums are SpermPrep™ TYB Media (ZDL Inc, Lexinngton, Ky.), media from IrvineScientific (Irvine, Calif.). A medium comprising an the egg yolk extender that may be modified by adjusting the pH and/or by adding a surfactant, as described in U.S. Pat. No. 6,593,309. Another medium that can be used is a medium containing LDL as described in Amirat et al., [Theriogenology, 61:895-907 (2004)]

Once sperm cells are at hand they are stained with acridine orange.

As mentioned hereinabove, “acridine orange” refers to an intercalating agent used for staining DNA that gives off a fluorescent emission varying between red and green, depending on the level of condensation i.e., single or double stranded).

It will be appreciated though that any detectable DNA interacting or intercalating dyes which are indicative of sperm DNA condensation by, for instance, level of emission or emission of a different wavelength, can be used in accordance with the present invention. Thus, different gating, or a different percentage of stained cells within the gate may be calculated for any such agents to characterize a staining pattern indicative of sperm cells with normal DNA condensation. Exemplary DNA fluorescent staining agents include but are not limited to Hoechst 33342, DAPI, Hoechst 33258, SYTOX Blue, Chromomycin A3, Mithramycin, YOYO-1, SYTOX Green, SYTOX Orange, Ethidium Bromide, 7-AAD, acridine orange, TOTO-1, TO-PRO-1, Thiazole Orange, Propidium Iodide (PI), TOTO-3, TO-PRO-3 and LDS 751.

Staining of the cells with acridine orange is typically done by incubating the sperm cells with the dye for a suitable period of time such that following washing; sperm cells are detected by the Flow Cytometer. Typically, incubation is effected for 30 minutes.

As mentioned hereinabove, the stained cells are analyzed by a Flow Cytometer, such as a laser scanning Cytometer. A Flow Cytometer typically consists of a laser light source, flow measurement chamber, and an optical system consisting of lenses, filters, and light detectors. Two photo-multiplier tubes (light detectors), one at 180 degrees and one at 90 degrees to the laser, are used to measure forward (FSC) and right-angle scatter (SSC), respectively. Three fluorescence detectors, each consisting of a filter and photomultiplier tube, are used to detect fluorescence. The three detectors sense green (FL1—530 nm), orange (FL2—585 nm), and red fluorescence (FL3—650 nm). Cells are identified by sort logic applied to all five of the detector signals (FSC, SSC, FL1, FL2, FL3) using a computer.

Exemplary Flow Cytometers that may be used in this aspect of the present invention are manufactured by companies such as Becton Dickinson (USA), Backman Coulter (USA), Partec (Germany) with the following specifications:

Laser excitation of 488 nm (blue laser, Argon laser)

Forward light Scatter FSC

Side light Scatter SSC

FL1—green filter—530 nm

FL2—orange filter—585 nm

FL3—red filter—650 nm.

Exemplary software that may be used to analyze the flow cytometry results include, but are not limited to those manufactured by:

Becton Dickinson—for instruments such as FacScan, FACSCalibur—CellQuest software,

Beckman Coulter—instruments such as Epics XL, and Cytomics FC 500,—Expo-32 or CXP software Partec—instruments such as PAS and CyFlow—FloMax software.

Preferably, analysis may be done with software—“Dyn-BioShaf Generator” (Dyn-Bioshaf, Caesarea, Israel), a Word macro based software application which provides automated analysis of test results. This automated report frees the user from having to manually calculate the final analysis results based on the values obtained by the Flow Cytometer. The above analysis is effected immediately following cell retrieval so as to minimize any external effect on the cell population of the semen sample, although the sample may be cryopreserved prior to analysis.

As shown in Example 1 in the Examples section, based on a survey of the emission results from 300 acridine orange stained sperm samples obtained from fertile men and 100 sperm samples analyzed as having an abnormal sperm's head morphology, specific staining patterns of sperm's DNA were analyzed by Flow Cytometer on two Dotplot graphs: one is FSC/SSC for gated sperm cells and the second FL1/FL3 that applied the gated sperm cells on the FSC/SSC gate. The novel gate was defined for “normal staining fluorescent emission”, characteristic of sperm with normal DNA condensation (described below), wherein DNA is labeled as abnormal when the packaging of sperm's chromatin is too condense and/or fragmented.

As mentioned above, the number of events gated as simultaneously being stained with a green fluorescence intensity higher than 100 nm and red fluorescence intensity in a range selected from 20-1010 nm, representing a cell population of the stained sperm cells is determined. The number of events in the gate is converted to the percentage of sperm cells falling within the gate and is indicative of the quality of the sperm cells.

According to a preferred embodiment of this aspect of the present invention, when the cell population is above 65% of the stained cells, the quality of the sperm cells is considered good.

As used herein, the phrase “normal DNA condensation” refers to the condensation level of DNA that allows the DNA to properly undergo fusion with the egg and create a functional zygote i.e., DNA which is not nicked, lose or too condensed. Morphologically, sperm with DNA which is too condensed will result in a smaller sperm head and sperm with DNA which is too loose will result in a larger sperm head, a characteristic defined as “swollen head”

As used herein “good quality sperm” refers to the ability of sperm cells to fertilize and optionally result in pregnancy, i.e., embryonic pulse is observed.

As shown in Example 2 in the Examples section that follows, 85% of sperm qualified as normal according to the methods of the present invention, exhibited a high rate of fertilization in IVF and 40.5% of fertilizations with sperm qualified as normal, ended in a positive pregnancy outcome.

As further described in Example 2 in the Examples section that follows, while reducing the present invention to practice, the present inventor has found that sperm cells located in the above gate can be used to improve pregnancy outcome which is a far more valuable clinical indicator than fertilization alone.

The methods of the present invention may be used for the routine evaluation of semen, e.g., for artificial insemination; determining commercial value, toxicology, e.g., in clinical studies or environmental influences on sperm quality, determining if sperm samples comprise sperm with an abnormal quantity of DNA i.e., sperm with numerical or structural chromosomal abnormalities, and the like.

Thus, the method described above is informative as to the quality of the sperm cell samples. The methods of the present invention can additionally be used for the enrichment of good quality sperm from a specific sperm sample.

Thus, according to another aspect of the present invention there is provided a method of collecting a sperm cell population. The method comprising staining sperm cells with acridine orange as described above; gating the above mentioned events and collecting the cell population of the stained sperm cells falling within the gate.

The cell population from the defined gate mentioned above may be directly sorted out of the general sperm sample into a tube or multi-well plate using the Flow Cytometer. Cells can then be used directly for fertilization, or can be cyropreserved for future use or analysis.

While sperm of varying DNA condensation can reach and fertilize the egg, only sperm with normal DNA condensation will be able to finalize the complex process of cell duplication and subsequent embryogenesis that follow fertilization.

Thus, according to yet another aspect of the present invention there is provided a method of improving pregnancy outcome. The method comprising, staining sperm cells as described above; gaiting the above mentioned events; collecting the cell population of the stained sperm cells falling within the gate as mentioned above; and using the collected cell population for fertilization.

As used herein, the term “fertilization” refers to the formation of a zygote as a result of contact between a male and a female germ cell.

It should be appreciated that for fertilization purposes the agent used for staining the cells should be non-harmful to the sperm cell, so as not to interfere with the formation of the zygote. Acridine orange is considered not hazardous according to criteria of Worksafe Australia. When used in dark conditions, acridine orange does not harm the cell [J. Frangois, et al., Investigative ophtomology 18: 599-605 (1976)], and is used in the art for in vivo staining [e.g., staining of rats in vivo in Masuda Y. et al., Jpn. J. Pharmacol. 77: 315-318 (1998); Use of acridine orange to assess lymphocyte migration in vivo in Popugailo M V et al., Bull Exp Biol Med 108: 949-951.

According to one embodiment of this aspect of the present invention fertilization may be effected by artificial insemination or in vitro fertilization (IVF). Preferably, artificial insemination may be by self insemination or intrauteirine insemination, wherein sperm is placed inside the uterus at the point of ovulation. In vitro fertilization may be by Intracytoplasmic Sperm Injection (ICSI), in which a sperm is injected directly into an egg to achieve fertilization, and the fertilized egg is then returned to the uterus.

Thus, the present invention provides methods for assessing sperm cell quality. It will be appreciated that in order to improve accuracy, control purposes and/or information collection other methods can be used for qualifying sperm cells in conjunction with the present metholodology. These methods include assessing the motility, viability, semen pH, sperm cell form, morphology, viscosity, presence or absence of pus cells, ability to undergo acrosome reaction and number of sperm cells in a sperm sample, as described in U.S. Pat. Application No. 20030190681.

The above mentioned guidelines and reagents may be included in a kit.

Thus, according to another aspect of the present invention there is provided a kit for qualifying sperm cells, the kit comprising acridine orange; and instructions for analyzing acridine orange stained sperm cells gated as simultaneously being stained with a green fluorescence intensity higher than 100 nm and red fluorescence intensity in a range selected from 20-1010 nm; gate obtained by FACS.

The kit of the present invention may, if desired, be presented in a pack which may contain one or more units of the kit of the present invention.

The pack may also be accommodated by a notice associated with the container in a form prescribed by a governmental agency regulating the manufacture, use or sale of laboratory supplements, which notice is reflective of approval by the agency of the form of the compositions.

As used herein the term “about” refers to ±10% and should be referred to as if precedes any number in the present specification.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Determination of an Acridine Orang DNA Staining Pattern Characteristic of Normal Sperm

A fixed Flow Cytometer staining gate was designed by evaluated sperm's DNA staining from fertile males with high quality semen sample.

Materials and Experimental Procedures

Use of Acridine Orang to assess DNA condensation level—Analysis of DNA condensation was done on sperm cells stained with the fluorescent reagent—acridine orange (Sigma-Aldrich Ltd, Rechovot, Israel). This reagent interacts with the sperm's head DNA by intercalation or electrostatic attractions. In condensed chromatin, the DNA is packed in a way that does not allow efficient acridine orange intercalation and the dye has green fluorescence. However, upon association with uncondensed DNA, the emission of this dye is shifted to the red fluorescence. Thus, a sperm cell with condensed DNA will emit a characteristic fluorescence lying between the red and green, which can be read by the Flow Cytometer.

Preparing sperm samples—Sperm cells were pelleted (5 minute centrifugation, 250 g), seminal plasma was discarded and pellet was washed in Ham's F-10 Buffer (2 ml; Biological Industries Ltd, Beit Haemek, Israel]. Cells were then resuspended in 2 ml clean buffer and the washing step was repeated twice. A 20 fold dilution was then made of each semen sample (0.2 ml of sample into 3.8 ml Buffer). Acridine orange solution (0.01 ml) was added to 1 ml of the diluted sample, and after 30 minutes of incubation (RT, dark conditions) the sample was washed (2 ml Buffer, and 5 min centrifugation at 250 g/1250 rpm, repeated twice) and read by Flow Cytometer (FacScan, Becton Dickenson, USA; CyFlow, Partec, Germany) supplied with Argon laser (488 nm), measuring emission of 530 nm (green light; FL1) and 650 nm (red light; FL3), analyzing size of particles (forward scatter/side scatter; FSC/SSC). Analysis was done with software-“Dyn-BioShaf Generator” (Dyn-Bioshaf, Caesarea, Israel), a Word macro based software application which provides automated analysis of test result. This automated report frees the user from having to manually calculate the final analysis results based on the values obtained by the Flow Cytometer.

Obtaining a ‘normal’ fluorescent emission staining gate characterized for normal DNA condensation—The “normal” DNA condensation status of sperm was assessed in a clinical study that carried out at HaEmek Medical Center (Afula, Israel—in 1999). Three hundred semen samples obtained from fertile males and categorized as having normal sperm morphology, and one hundred semen samples analyzed as having an abnormal sperm's head morphology, according to the latest manual of the W.H.O [World Health Organization. Laboratory manual for the examination of human semen and sperm-cervical mucus interaction. 4^{th} ed. New York: Cambridge University Press, 1999] were analyzed in this study. Based on the Acridin Orange staining pattern of sperm's DNA, a characterized gate for “normal staining fluorescent emission” was designed.

The DNA condensation test assay—A reading frame characterized with the gate described herein above was applied for reading by the Flow Cytometer. A cut-off value of 65% was applied to define a sample with normal DNA condensation. Samples were thereby classified as normal by having a staining pattern with 65% of events within the gate while abnormal samples were classified as such if 35% and more of the samples were situated outside of the gate.

Accuracy test—The accuracy of the cut-off value (normal/abnormal) was demonstrated by the shift of the normal staining pattern as a result of pre treatment of sperm cells with low concentration DNAase I (1 U/ul; 10 ul/ml) or by high temperature (75° C., 30 minutes) that influence DNA condensation. The staining pattern of tested semen samples was compared to the defined “normal staining fluorescent emission” gate.

Reproducibility studies—Semen samples are extremely labile, therefore reproducibility studies must necessarily be limited to those which can be performed within a short period of time. Thus, only with-in run and operator-to-operator reproducibility studies were performed. In order to demonstrate the within-run reproducibility of the DNA condensation assay sixty-four semen samples were run in triplicate at the Boston IVF Center (Waltham, USA) producing three DNA condensation results for each sample. The data were analyzed by a one-way analysis of variance to estimate the average within-sample variability and the intra-class correlation. In order to test the operator-to operator reproducibility, ten semen samples were each tested by three different technicians (Dyn-BioShaf, Nesher, Israel) and the results compared and tested for differences among the three means. The three assays by the different technicians were then treated as random determinations and the data were analyzed by a one-way analysis of variance.

Results

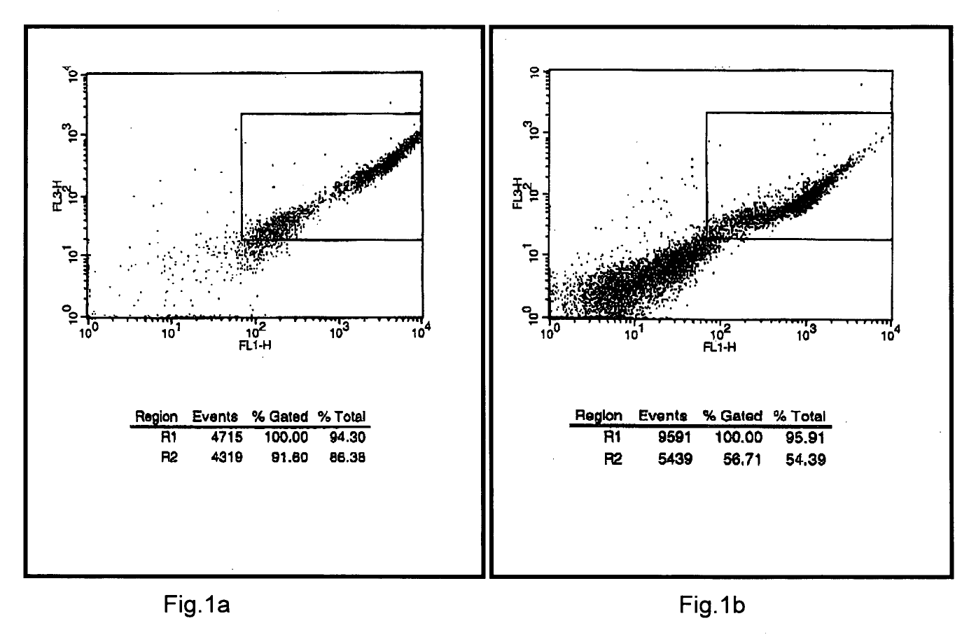

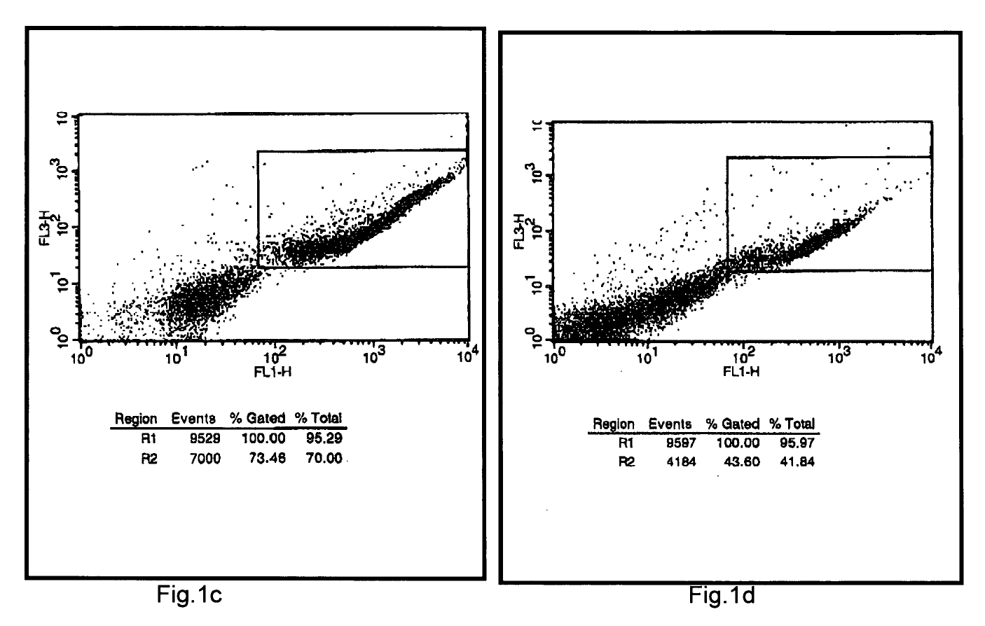

Acuracy of DNA condensation test—FIGS. 1a-d demonstrate the difference in staining between a normal sperm DNA condensation staining pattern (FIGS. 1a and c) and an abnormal staining pattern (FIGS. 1b and d). In the normal sperm sample, more than 65% of events are located in the predetermined gate, while in the abnormal (FIG. 1b), or treated sample (FIG. 1d), less than 35% of the events are located in this gate.

Reproducibility studies—Variability test results have shown the between patient variance to be 2.483 and the within patient variance to be 3.88. Hence, the intra-class correlation of semen samples was found to be 0.985. This suggests excellent agreement among repeat assays on the same patient sample. Moreover, the average within patient standard deviation (SD-square root of the within patient variance) was found to be 1.97 and the within patient coefficient of variation—was 2.4%.

When comparing results between technicians (operator to operator variability) the mean results of semen sample tests for the three technicians were 66.6, 66.9, and 67.6 (test for differences among the three means, p=0.15), suggesting no differences among the different technicians. When the three assays by the different technicians were treated as random determinations and the data were analyzed by a one-way analysis of variance, the resulting within-patient standard deviation was found to be 1.16 and the CV was 1.7%. This suggests that different technicians performing the assay do not add variability to the results, again pointing to the reproducibility of the assay.

### Example 2

### Clinical Study

The connection between DNA condensation and fertilization and pregnancy outcome was evaluated in a clinical study carried on 485 semen samples.

Materials and Experimental Procedures

GSA kit—The General sperm analysis kit (GSA kit; Dyn-Bioshaf, Caesarea, Israel) uses flow cytometry to provide automated analysis of sperm count, motility, vitality, sperm's head morphology, which is indicative of DNA condensation, and the presence of white blood cells.

Clinical trial—A clinical trial was carried out in four USA clinics and one Israeli clinic; 485 semen samples were evaluated with the GSA kit for normal sperm head morphology which is indicative for DNA condensation. The following clinics participated in the trial:

1. Houston Tex. USA—Houston Medical Center, Baylor College of Medicine; N=56 semen samples.

2. Carmel Medical Center—Haifa, Israel; N=89 semen samples.

3. Boston IVF, Waltham Mass., USA; N=157 semen samples.

4. Stanford IVF REI, Palo Alto, Calif., USA; N=63 semen samples.

5. Atlanta—RBA, GA, USA N=120 semen samples.

Routine Analysis for Morphology was performed according to the W.H.O manual [World Health Organization. Laboratory manual for the examination of human semen and sperm-cervical mucus interaction. 4^{th} ed. New York: Cambridge University Press, 1999]. Briefly, an aliquot of semen was placed on a slide and was then smeared with a second slide (two smears from each sample). Smears were air dried and immediately fixed (15 minutes, 95% Ethanol). Smears were stained with Haematoxylin, OG6 and EA50 (PolyScientific—Bayshore, N.Y., USA) and were then mounted and dried overnight. Duplicate counts of 200 sperm cells were assessed in each slide using a light microscope with a total magnification 1000× using a high qualitative 100× non-phase contrast objective under immersion oil and correctly adjusted bright field. Evaluation of sperm's morphology was done according to the W.H.O guide using a report form. Sperm's morphology was considered as abnormal if defects were defined in one or more parts of the sperm cell (e.g head, neck/mid-piece, tail, cytoplasmic droplets). As based on strict criteria for sperm's morphology evaluation, the normal forms considered alone are called the ‘percentage of ideal forms’ (PIF). A PIF greater than 4% is considered favorable and less than 4% unfavorable. At least 200 cells per slide are to be evaluated [Kruger, T. F et al.,. Fertil. Steril. 49, 112-117 (1988)]. The cut-off for normal morphology method used was >3% normal forms in Atlanta—RBA and >4% normal forms in the rest of the centers.

The Routine Analysis morphology results for normal/abnormal were then compared to the Flow Cytometer head morphology results effected with the GSA kit for normal/abnormal samples. Chi test X² was applied for evaluating statistically significant associations between the two parameters (DNA condensation/Routine Analysis morphology). The results obtained by both methods were compared and an overall agreement rate was determined

Results

Agreement between sperm morphology tests by Routine Analysis and Flow Cytometer tests of sperm's head morphology with the GSA kit—Table 1 below presents an agreement study between the GSA kit, in which the head morphology analysis is indicative of DNA condensation and Routine Analysis of sperm morphology tests, on the results of semen analysis done in the 5 centers mentioned in the Methods section. Results show the agreement of the GSA Kit with the Routine Analysis—for abnormal sperm morphology was 83.9%, the agreement of the routine analysis—for normal sperm morphology with THE GSA Kit was 87.8%, and the overall agreement rate was 85.6% (exact 95% confidence interval, 82.1%-88.6%).

TABLE 1

GSA Kit

| Routine Analysis | Normal | Abnormal | Total |
|---|---|---|---|
| Normal | 180 | 25 | 205 |
| | 87.8% | 12.2% | |
| Abnormal | 45 | 235 | 280 |
| | 16.1% | 83.9% | |
| Total | 225 | 260 | 485 |

P < 0.001 - Significant association

The association between Routine Analysis sperm morphology and DNA condensation can be attributed to the influence of the DNA condensation state on the shape and size of the sperm head. The majority of sperm morphology defects are located in the sperm head [El-Ghobashy A A. and West C R, J Andro 24(2):232-238 (2003)], therefore the association between the Routine Analysis morphology test results and the DNA condensation parameter is expected. However, since the Routine Analysis test considers size and shape changes observed in different parts of the sperm cell, apart from the sperm head, these differences contribute to the incomplete agreement between these two parameters.

Agreement between technicians testing the same samples by Routine Analysis—At two clinics (Houston Tex. USA—Houston Medical Center, Baylor College of Medicine and Carmel Medical Center—Haifa, Israel) the morphology of the same N=121 semen samples was evaluated by two independent technicians following the Routine Analysis and the agreement rates between the results obtained by the two technicians were determined. Results are given in Table 2 below, agreement rate being 74.5%.

TABLE 2

Routine Analysis 2

| Routine Analysis 1 | Normal | Abnormal | Total |
|---|---|---|---|
| Normal | 48 | 18 | 66 |
| Abnormal | 13 | 42 | 55 |
| Total | 61 | 60 | 121 |

Overall Agreement = 74.4%

Association between DNA condensation, fertilization (IVF) and pregnancy rate—The association between DNA condensation and fertilization by in vitro fertilization (IVF; conventional) or pregnancy rate was determined in Boston IVF, and is summarized below, in Table 3 and Table 4, respectively.

TABLE 3

| | Low rate | High rate | Total |
|---|---|---|---|
| Abnormal DNA | 9 (34.6%) | 17 (65.4%) | 26 |
| Normal DNA | 17 (14%) | 104 (85.6%) | 121 |
| Total | 26 | 121 | 147 |

0.05 < P < 0.01 - Significant association

TABLE 4

| Pregnancy | No | Yes | Total |
|---|---|---|---|
| Abnormal DNA | 16 (84.2%) | 3 (15.8%) | 19 |
| Normal DNA | 72 (59.5%) | 49 (40.5%) | 121 |
| Total | 88 | 52 | 140 |

0.05 < P < 0.01 Significant association

The impact of DNA condensation/fragmentation status on fertility potential is well known [e.g., Ibrahim M E, et al., Arch Androl 21:129-3 (1988); Spano M, et al., 73:43-50 (2002)]. Association between abnormal morphology and low fertilization rate [e.g., Hammadeh M E et al., Andrologia 30(1):29-35. (1998)] is also well established, and can be attributed in most cases to the influence of the DNA condensation status on sperm head's morphology. Indeed, the results presented in the present invention show a statisticaly significant association between DNA condensation and fertilization and pregnancy rate in IVF.

Results have also shown that the negative predictor value of abnormal DNA condensation on fertilization rate is low (0.346). Only in 34.6% (9/26) of cases with abnormal DNA condensation the fertilization rate was low. However, while DNA condensation is a low predictor of fertilization rate, results show it is an excellent predictor for pregnancy outcome of IVF treatment. In this case the negative predictor value of abnormal DNA condensation is high. Results show that 84.2% (16/19) cases with abnormal DNA did not achieve pregnancy. Thus, DNA condensation was found by the present invention to be a better predictor of fertilization outcome (i.e. pregnancy) which is more associated with DNA quality, while the traditional assessment of overall semen cell morphology has more to do with fertilization alone.

Given together, results display the DNA condensation test as a simple and reliable assay that can be easily and routinely used by the clinical laboratory technician in fertility laboratories as a diagnostic parameter for the evaluation of sperm fertility potential.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. A method of assessing fertilization ability of sperm cells, the method comprising:
(a) staining the sperm cells with acridine orange so as to obtain stained sperm cells; and
(b) using a fluorescence activated cell sorter (FACS), gating events simultaneously stained with a green fluorescence intensity higher than 100 and red fluorescence intensity of 20-1010 said events representing a cell population of said stained sperm cells, wherein a percentage of said cell population is indicative of the fertilization ability of the sperm cells.

2. The method of claim 1, wherein when said cell population is above 65% of said stained sperm cells, the sperm cells have fertilization ability.

3. The method of claim 1, wherein said cell population is characterized by a normal DNA condensation pattern.

4. The method of claim 1, wherein the sperm cells are of a mammalian semen sample.

5. The method of claim 1, wherein the sperm cells are of a human semen sample.

6. A method of collecting a sperm cell population, the method comprising:
(a) staining sperm cells with acridine orange so as to obtain stained sperm cells;
(b) using a fluorescence activated cell sorter (FACS), gating events simultaneously stained with a green fluorescence intensity higher than 100 and red fluorescence intensity of 20-1010, said events representing a cell population of said stained sperm cells; and
(c) collecting said cell population of said stained sperm cells.

7. The method of claim 6, wherein said cell population is characterized by a normal DNA condensation pattern.

8. The method of claim 6, wherein the sperm cells are of a mammalian semen sample.

9. The method of claim 6, wherein the sperm cells are of a human semen sample.

10. An isolated sperm cell population generated according to the method of claim 6.

11. A method of improving pregnancy outcome, the method comprising:
(a) staining sperm cells with acridine orange so as to obtain stained sperm cells;
(b) using a fluorescence activated cell sorter (FACS), gating events simultaneously stained with a green fluorescence intensity higher than 100 and red fluorescence intensity of 20-1010, said events representing a cell population of said stained sperm cells; and
(c) collecting said cell population of said stained sperm cells; and
(d) using said cell population of step c for fertilization, thereby improving pregnancy outcome.

12. The method of claim 11, wherein said cell population is characterized by a normal DNA condensation pattern.

13. The method of claim 11, wherein said fertilization is effected by a method selected from the group consisting of artificial insemination and in vitro fertilization (IVF).

14. The method of claim 11, wherein the sperm cells are of a mammalian semen sample

15. The method of claim 11, wherein the sperm cells are of a human semen sample.

16. A kit for assessing fertilization ability of sperm cells, the kit comprising
(i) acridine orange; and
(ii) instructions for analyzing acridine orange stained sperm cells simultaneously stained with a green fluorescence intensity higher than 100 and red fluorescence intensity of 20-1010, gated by FACS.
